(19) United States
(12) Patent Application Publication
(10) Pub. No.: US 2006/0171990 A1
(43) Pub. Date: Aug. 3, 2006

(54) Drug delivery materials made by sol/gel technology

(76) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(21) Appl. No.: 11/346,983

(22) Filed: Feb. 3, 2006

Related U.S. Application Data
(60) Provisional application No. 60/649,927, filed on Feb. 3, 2005.

(51) Int. Cl. A61F 2/00; B01J 13/04

(52) U.S. Cl. 424/426; 264/4.1

(57) ABSTRACT

The present invention relates to a method of producing a drug delivery material by encapsulating a biologically or therapeutically active agent in a shell, combining the encapsulated agent with a sol, and converting the combination into a solid or semi-solid drug delivery material. The present invention further relates to drug delivery materials produced by this exemplary method, and to implants formed at least in part from these materials.

### CROSS-REFERENCE TO RELATED APPLICATION(S)

The present application claims priority from U.S. Patent Application Ser. No. 60/649,927, filed Feb. 3, 2005, the entire disclosure of which is incorporated herein by reference.

### FIELD OF THE INVENTION

The present invention relates to drug delivery materials comprising a biologically or therapeutically active compound and an encapsulating shell, which may be incorporated in a matrix prepared by sol/gel technology, and which may be suitable for use in implants.

### BACKGROUND OF THE INVENTION

Materials being implanted into the human or animal body must have certain bio-chemical properties to avoid unwanted side-effects such as inflammatory tissue responses, intolerance reactions, immune reactions and the like, which may be caused by chemical and/or physical irritations. Implant materials should be bio-compatible and non-toxic, and preferbly may be used for a variety of different applications requiring a wide range of various properties. Implant materials used for, e.g., medical implants such as surgical and/or orthopaedic screws, plates, joint prostheses, artificial heart-valves, vascular prostheses or stents, as well as for subcutaneously or intramuscularly implantable active agent depots, may require biocompatible materials having sufficient mechanical strength. Strength may be particularly important if support of tissue is required, for example, in the case of stents or bone implants, whereas implant materials may also require bio-active properties such that surrounding tissue can form an interfacial bond with the implant. For implantable active agent depots, use of materials that are dissolvable in the presence of physiological fluids or can be slowly bioerodible may be preferred.

Sol/gel-process technology can be widely applied to build up different types of networks. The linkage of components forming the sol or gel can take place in several ways, e.g. via hydrolytic or non-hydrolytic sol/gel-processing.

A “sol” may be understood to refer to a dispersion of colloidal particles in a liquid, and the term “gel” may connote an interconnected, rigid network of pores of submicrometer dimensions and polymeric chains whose average length is typically greater than a micrometer. A sol/gel-process may comprise mixing of precursors, e.g. sol/gel forming components, to form a sol, adding further additives or materials, casting the mixture in a mold or applying the sol onto a substrate in the form of a coating, gelation of the mixture, whereby the colloidal particles can be linked together to become a porous three-dimensional network, aging of the gel to increase its strength; converting the gel into a solid material by drying and/or dehydration or chemical stabilisation of the pore network, and densification of the material to produce structures with ranges of physical properties. Such processes are described, for example, in Henge and West, The Sol/Gel-Process, 90 Chem. Ref. 33 (1990).

The term “sol/gel” as used within this specification may include either a sol or a gel. A sol can be converted into a gel using processes including those described above, e.g., by aging, curing, raising of pH, evaporation of solvent, or any other conventional methods.

The term “semi-solid” can refer to materials having a gel-like consistency, i.e., materials that may be dimensionally stable at room temperature, but which can have a certain elasticity and flexibility, typically due to a residual solvent content.

Among the implant materials that can possess sufficient variability of intrinsic properties, bio-active glasses or glass ceramics made by sol/gel process technology may be suitable for the production of, for example, support implants, drug delivery depots, or load-bearing synthetic grafts. Bio-active glasses and glass ceramics having certain compositions may undergo surface corrosion reactions when exposed to body fluids, or may even be fully bioerodible or dissolvable in the presence of physiological fluids.

International Patent Publication WO 96/03117 describes carriers comprising silica-based glass that can provide a controlled release of biologically active molecules, and methods of preparing them. The carriers described therein can be prepared using a sol/gel derived process, and biologically active molecules such as, e.g., antibiotics or proteins can be incorporated in the matrix of the glass during the production process. The release rate of the bio-active molecules in these materials can be controlled by controlling the micro-porosity of the sol/gel glasses through variations in the water content, the addition of acids, and aging and drying times. Controlled release of active agents can be achieved by the controllable micro-porosity of such bio-active sol/gel derived glasses. However, although the release of active agents may be delayed in these materials, the actual release rate of the active agents is not well-controlled and may exhibit large fluctuations, which can lead to adverse side effects with some agents.

European Patent Application No. EP 0 680 753 A2 describes a sol/gel derived silica material containing a biologically active substance such as a therapeutically active agent, where the release rate of the active agent is controlled by the addition of penetration enhancers such as polyethylene glycol or sorbitol, or the addition of other modifying agents which can enhance the release of the active agent by either aiding dissolution via swelling processes or by inhibiting diffusion in order to modify the permeability of the matrix. Modifying agents which may be used for more precise adjustment of release rates of the active agents can include, for example, water-soluble substances such as sugars or salts of organic acids, which may accelerate the release rate of active agents from the matrix because of their solubility in body fluids, which may lead to their dissolution and and thus increase the permeability of the sol/gel-produced matrix. Other modifying agents that increase the permeability of the matrix in the presence of body fluids may include polyanionic compounds such as salts of polystyrene, sulfonic acid, polyacrylic acids, carboxymethyl celluloses, dextrane sulphate or cellulose sulphate, and the like. Each of these release-modifying agents accelerates the release of the active agent. However, such multi-component systems can be rather complex and costly, and it may be very difficult to reliably and reproducibly adjust the release rate of the active agent with the use of penetration adjuvants and modifyers.

Thus there is a need for bio-compatible drug delivery materials which may be produced as coatings or bulk materials, especially for the production of implants or coated implants, which can reliably and reproducibly provide an adjustable controlled release of an active agent incorporated therein.

### SUMMARY OF THE INVENTION

Therefore, it is one of the objects of the present invention to provide drug delivery materials which are easily producible at low cost. A further object of the present invention is to provide drug delivery materials that permit a controlled and reproducible release of the active agent incorporated therein. Another object of the present invention is to provide controlled-release delivery materials suitable for the production of medical implants. A still further object of the present invention is to provide controlled-release drug delivery materials which may be used for coating of medical implants such as aortic valves or stents and the like. Yet another object of the present invention is to provide a process which avoids detrimental interactions of the active agents with the sol/gel materials, allowing for incorporation of sensitive drugs in a sol/gel matrix without deactivating or adversely affecting the active agent.

Therefore, it is one of the objects of the present invention to provide a drug delivery material which provides a controlled release of the active agents and which optionally may be controllably dissolvable or bioerodible. It is a further object of the present invention to provide a process for manufacturing such delivery materials which comprises the steps of encapsulating at least one biologically or therapeutically active agent in a shell and combining the encapsulated active compound with a sol, followed by converting the resulting combination into the inventive drug delivery material.

It is yet another object of the present invention to provide a process for the manufacture of drug delivery materials, the process comprising the steps of encapsulating at least one biologically and/or therapeutically active agent in a shell, combining the encapsulated active compound with sol and converting the resulting combination into a solid or semi-solid material.

These and other objects may be achieved by certain exemplary embodiments of the present invention which may provide solid drug delivery materials comprising biologically or therapeutically active agents encapsulated in a shell, and which are further incorporated in a sol/gel matrix.

In one exemplary embodiment of the present invention, a process can be provided for the manufacture of a drug delivery material, wherein a biologically or therapeutically active compound is first encapsulated in a polymeric shell before being combined with a sol. The biologically or therapeutically active compound can be a therapeutic agent taht is capable of providing a direct or indirect therapeutic, physiological and/or pharmacological effect in a human or animal organism. Preferred biologically or therapeutically active compounds may include, e.g., are medicaments, drugs, pro-drugs, or targeting groups and the like. The sol used for preparing the drug delivery material may be formed in a hydrolytic or non-hydrolytic sol/gel process. Bioresorbable polymers and biopolymers may be especially preferred for encapsulating the active agents in a polymer shell.

In certain exemplary embodiments of the present invention, the material produced may be dissolvable when exposed to physiological fluids or have bioerodible properties in the presence of such fluids. These materials may also provide a sustained or controlled release of the active agent when inserted into the human or animal body.

In another exemplary embodiment of the present invention, the drug delivery material may be used to coat stents or other medical implants.

These and other embodiments of the present invention are described by or encompassed by the detailed description provided herein.

### DETAILED DESCRIPTION OF EXEMPLARY EMBODIMENTS

Sol/gel technology can allow for the production of highly biocompatible and/or bioerodible, materials at low temperatures. Sol/gel derived materials may form suitable matrices for drug delivery materials or coatings, and a combination of a sol/gel derived matrix with polymer encapsulated drugs incorporated therein can provide controlled-release materials with adjustable release characteristics for a wide variety of biomedical applications.

Drug delivery materials produced in accordance with certain exemplary embodiments of the present invention may exhibit the advantageous property that they can be easily and reproducibly processed at low temperature from sols and/or gels. In particular, sols/gels and combinations thereof may be suitable for coating of substrates with porous or non-porous drug delivery film coatings. Coatings as well as shaped bulk drug delivery materials can be obtained in accordance with the exemplary embodiments of the methods of the present invention.

In exemplary embodiments of the present invention, biologically and/or therapeutically active agents (“active agents” or “active compounds”) may first be encapsulated in a polymer material. Active agents suitable for being encapsulated and incorporated into the drug delivery material may include therapeutically active agents which are capable of providing direct or indirect therapeutic, physiological and/or pharmacological effect in a human or animal organism. Therapeutically active agents may include, but are not limited to, conventional medicaments, drugs, pro-drugs, targeting groups, or a drug or pro-drug comprising a targeting group.

In one exemplary embodiment of the present invention, the active agent may be a compound used for agricultural purposes such as, for example a fertilizer, pesticide, microbicide, herbicide, algicide and the like.

The active agents may be in crystalline, polymorphous or amorphous form or any combination thereof. Suitable therapeutically active agents may include, e.g., enzyme inhibitors, hormones, cytokines, growth factors, receptor ligands, antibodies, antigens, ion binding agents such as crown ethers and chelating compounds, substantially complementary nucleic acids, nucleic acid binding proteins including transcriptions factors, toxines and the like. Examples of active agents include, for example, cytokines such as erythropoietine (EPO), thrombopoietine (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine, and further active agents such as those described in Physician's Desk Reference, 58^{th} Edition, Medical Economics Data Production Company, Montvale, N.J., 2004 and the Merck Index, 13^{th} Edition, including those listed on pages Ther-1 to Ther-29.

In a preferred exemplary embodiment of the present invention, the therapeutically active agent may be selected from the group of drugs used for the therapy of oncological diseases and cellular or tissue alterations. Suitable therapeutic agents can include, e.g., antineoplastic agents, including alkylating agents such as alkyl sulfonates, e.g., busulfan, improsulfan, piposulfane, aziridines such as benzodepa, carboquone, meturedepa, uredepa; ethyleneimine and methylmelamines such as altretamine, triethylene melamine, triethylene phosphoramide, triethylene thiophosphoramide, trimethylolmelamine; so-called nitrogen mustards such as chlorambucil, chlomaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, and the like, as well as combinations and/or derivatives of any of the foregoing.

In a further exemplary embodiment of the present invention, the therapeutically active agent may be selected from the group comprising anti-viral and anti-bacterial agents such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsaure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, and the like, as well as combinations and/or derivatives of any of the foregoing.

In a still further exemplary embodiment of the present invention, the therapeutically active agent may comprise radio-sensitizer drugs, steroidal or non-steroidal anti-inflammatory drugs, or agents referring to angiogenesis, such as, e.g., endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 and the like, as well as combinations and/or derivatives of any of the foregoing.

In another exemplary embodiment of the present invention, the therapeutically-active agent may be selected from the group comprising nucleic acids, wherein the term nucleic acids further comprises oliogonucleotides wherein at least two nucleotides may be covalently linked to each other, for example, to provide gene therapeutic or antisense effects. Nucleic acids may comprise phosphodiester bonds, which can include those which are analogs having different backbones. Analogs may also contain backbones such as, for example, phosphoramide as described in, for example, Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986); phosphorothioate as described in, for example, Mag et al., Nucleic Acids Res. 19:1437 (1991); and U.S. Pat. No. 5,644,048, phosphorodithioate as described in, for example, Briu et al., J. Am. Chem. Soc. 111:2321 (1989), O-methylphosphoroamidit-compounds (see, e.g., Eckstein, Oligonucleotides and Analogs: A Practical Approach, Oxford University Press), and peptide-nukleic acid-backbones and their compounds as described in, for example, Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996). Further analogs may include those having ionic backbones as described in, for example, Denpcy et al., Proc. Natl. Acad. Sci. USA 92:6097 (1995), or non-ionic backbones as described in, for example, U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research,” Ed. Y. S. Sanghui and P. Dan Cook. The nucleic acids having one or more carbocylic sugars may also be suitable as nucleic acids for use in exemplary embodiments of the present invention, such as those described in Jenkins et al., Chemical Society Review (1995), pages 169-176 and in Rawls, C & E News, 2 Jun. 1997, page 36. In addition to conventional nucleic acids and nucleic acid analogs, mixtures of naturally occurring nucleic acids and nucleic acid analogs or mixtures of nucleic acid analogs may also be used.

In a further exemplary embodiment of the present invention, the therapeutically active agent may comprise one or more metal ion complexes, such as those described in International Patent Applications PCT/US95/16377, PCT/US95/16377, PCT/US96/19900, and PCT/US96/15527, wherein such agents may reduce or inactivate the bioactivity of their target molecules, including proteins such as enzymes.

Therapeutically active agents may also be anti-migratory, anti-proliferative or immune-supressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, VEGF, statines and the like, as well as their derivatives and analogs.

Other active agents or components of active agents may include, e.g., heparin, synthetic heparin analogs (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid (i.e. aspirin), ticlopidine, clopidogrel, abciximab, dextrans; corticosteroids such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs) such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics such as alkaloides and podophyllum toxins such as vinblastine, vincristine; alkylating agents such as nitrosoureas, nitrogen lost analogs; cytotoxic antibiotics such as daunorubicin, doxorubicin and other anthracyclines and related substances, bleomycin, mitomycin; antimetabolites such as folic acid analogs, purine analogs or pyrimidine analogs; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenyloin, tocainid; class Ic antiarrhythmics, e.g., propafenon, flecainid(acetate); class II antiarrhythmics beta-receptor blockers such as metoprolol, esmolol, propranolol, metoprolol, atenolol, oxprenolol; class III antiarrhythmics such as amiodarone, sotalol; class IV antiarrhythmics such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers such as prazosin, urapidil, doxazosin, bunazosin, terazosin, indoramin; vasodilatators such as dihydralazine, diisopropylamine dichloracetate, minoxidil, nitroprusside sodium; other antihypertensives such as indapamide, co-dergocrine mesylate, dihydroergotoxin methanessulfonate, cicletanin, bosentan, fludrocortisone; phosphodiesterase inhibitors such as milrinon, enoximon and antihypotensives such as in particular adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGFβ, PDGF, VEGF, bFGF, TNFα, NGF, GM-CSF, IGF-a, IL-1, IL-8, IL-6, growth hormone; as well as adhesive substances such as cyanoacrylates, beryllium, silica; and growth factors such as erythropoetin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particulary recombinant BMPs such as recombinant human BMP-2 (rhBMP-2), bisphosphonate (e.g., risedronate, pamidronate, ibandronate, zoledronic acid, clodronsäure, etidronsäure, alendronic acid, tiludronic acid), fluorides such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as in particular β-lactam antibiotics, e.g., β-lactamase-sensitive penicillins such as benzyl penicillins (penicillin G), phenoxymethylpenicillin (penicillin V); β-lactamase-resistent penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscamet, inosine-(dimepranol-4-acetamidobenzoate), valganciclovir, valaciclovir, cidofovir, brivudin; antiretroviral active ingredients (nucleoside analog reverse-transcriptase inhibitors and derivatives) such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In accordance with certain exemplary embodiments of the present invention, the active agents such as those described above may be encapsulated in a polymeric shell or in vesicles, liposomes, micelles or the like. The encapsulation of the active agents by polymers may be achieved by various conventional polymerization techniques such as, e.g., dispersion-, suspension- or emulsion-polymerization. Preferred encapsulating polymers may include biopolymers as described herein below, or acrylic polymers such as polymethylmethacrylate (PMMA) or other latex-forming polymers.

The resulting polymer capsules, which contain the active agents, can further be optionally modified, for example by crosslinking the capsules and/or further encapsulation with several shells of polymer. Techniques to modify the polymers, if necessary, are well known to those skilled in the art, and may be employed depending on the requirements of the individual composition to be used in the inventive process. The use of encapsulated active agents prevents aggregation and the encapsulated active agents can be uniformly distributed in a sol/gel process without agglomerating.

Encapsulation of the active agents can produce covalently or non-covalently encapsulated active agents, depending on the individual materials used. For combining with the sol, the encapsulated active agents may be provided in the form of polymer spheres, particularly microspheres, or in the form of dispersed, suspended or emulgated particles or capsules. Conventional methods suitable for providing or manufacturing encapsulated active agents, dispersions, suspensions, emulsions, or mini-emulsions thereof may be utilized. Suitable encapsulation methods are described, for example, in Australian Patent Application No. AU 9169501, European Patent Publication Nos. EP 1205492, EP 1401878, EP 1352915 and EP 1240215, U.S. Pat. No. 6,380,281, U.S. Patent Publication No. 2004192838, Canadian Patent Publication No. CA 1336218, Chinese Patent Publication No. CN 1262692T, British Patent Publication No. GB 949722, and German Patent Publication No. DE 10037656; and in S. Kirsch, K. Landfester, O. Shaffer and M. S. El-Aasser, “Particle morphology of carboxylated poly-(n-butyl acrylate)/(poly(methyl methacrylate) composite latex particles investigated by TEM and NMR,” Acta Polymerica 1999, 50, 347-362; K. Landfester, N. Bechthold, S. Förster and M. Antonietti, “Evidence for the preservation of the particle identity in miniemulsion polymerization,” Macromol. Rapid Commun. 1999, 20, 81-84; K. Landfester, N. Bechthold, F. Tiarks and M. Antonietti, “Miniemulsion polymerization with cationic and nonionic surfactants: A very efficient use of surfactants for heterophase polymerization” Macromolecules 1999, 32, 2679-2683; K. Landfester, N. Bechthold, F. Tiarks and M. Antonietti, “Formulation and stability mechanisms of polymerizable miniemulsions,” Macromolecules 1999, 32, 5222-5228; G. Baskar, K. Landfester and M. Antonietti, “Comb-like polymers with octadecyl side chain and carboxyl functional sites: Scope for efficient use in miniemulsion polymerization,” Macromolecules 2000, 33, 9228-9232; N. Bechthold, F. Tiarks, M. Willert, K. Landfester and M. Antonietti, “Miniemulsion polymerization: Applications and new materials” Macromol. Symp. 2000, 151, 549-555; N. Bechthold and K. Landfester: “Kinetics of miniemulsion polymerization as revealed by calorimetry,” Macromolecules 2000, 33, 4682-4689; B. M. Budhlall, K. Landfester, D. Nagy, E. D. Sudol, V. L. Dimonie, D. Sagl, A. Klein and M. S. El-Aasser, “Characterization of partially hydrolyzed poly(vinyl alcohol). I. Sequence distribution via H-1 and C-13-NMR and a reversed-phased gradient elution HPLC technique,” Macromol. Symp. 2000, 155, 63-84; D. Columbie, K. Landfester, E. D. Sudol and M. S. El-Aasser, “Competitive adsorption of the anionic surfactant Triton X-405 on PS latex particles,” Langmuir 2000, 16, 7905-7913; S. Kirsch, A. Pfau, K. Landfester, O. Shaffer and M. S. El-Aasser, “Particle morphology of carboxylated poly-(n-butyl acrylate)/poly(methyl methacrylate) composite latex particles,” Macromol. Symp. 2000, 151, 413-418; K. Landfester, F. Tiarks, H.-P. Hentze and M. Antonietti, “Polyaddition in miniemulsions: A new route to polymer dispersions,” Macromol. Chem. Phys. 2000, 201, 1-5; K. Landfester, “Recent developments in miniemulsions—Formation and stability mechanisms,” Macromol. Symp. 2000, 150, 171-178; K. Landfester, M. Willert and M. Antonietti, “Preparation of polymer particles in non-aqueous direct and inverse miniemulsions,” Macromolecules 2000, 33, 2370-2376; K. Landfester and M. Antonietti, “The polymerization of acrylonitrile in miniemulsions: ‘Crumpled latex particles’ or polymer nanocrystals,” Macromol. Rapid Comm. 2000, 21, 820-824; B. z. Putlitz, K. Landfester, S. Forster and M. Antonietti, “Vesicle forming, single tail hydrocarbon surfactants with sulfonium-headgroup,” Langmuir 2000, 16, 3003-3005; B. z. Putlitz, H.-P. Hentze, K. Landfester and M. Antonietti, “New cationic surfactants with sulfonium-headgroup,” Langmuir 2000, 16, 3214-3220; J. Rottstegge, K. Landfester, M. Wilhelm, C. Heldmann and H. W. Spiess, “Different types of water in film formation process of latex dispersions as detected by solid-state nuclear magnetic resonance spectroscopy,” Colloid Polym. Sci. 2000, 278, 236-244; M. Antonietti and K. Landfester, “Single molecule chemistry with polymers and colloids: A way to handle complex reactions and physical processes?” ChemPhysChem 2001, 2, 207-210; K. Landfester and H.-P. Hentze, “Heterophase polymerization in inverse systems,” in Reactions and Synthesis in Surfactant Systems, J. Texter, ed.; Marcel Dekker, Inc., New York, 2001, pp 471-499; K. Landfester, “Polyreactions in miniemulsions,” Macromol. Rapid Comm. 2001, 896-936; K. Landfester, “The generation of nanoparticles in miniemulsion,” Adv. Mater. 2001, 10, 765-768; K. Landfester, “Chemie—Rezeptionsgeschichte” in Der Neue Pauly—Enzyklopädie der Antik, Verlag J. B. Metzler, Stuttgart, 2001, vol. 15; B. z. Putlitz, K. Landfester, H. Fischer and M. Antonietti, “The generation of ‘armored latexes’ and hollow inorganic shells made of clay sheets by templating cationic miniemulsions and latexes,” Adv. Mater. 2001, 13, 500-503; F. Tiarks, K. Landfester and M. Antonietti, “Preparation of polymeric nanocapsules by miniemulsion polymerization,” Langmuir 2001, 17, 908-917; F. Tiarks, K. Landfester and M. Antonietti, “Encapsulation of carbon black by miniemulsion polymerization,” Macromol. Chem. Phys. 2001, 202, 51-60; F. Tiarks, K. Landfester and M. Antonietti, “One-step preparation of polyurethane dispersions by miniemulsion polyaddition,” J. Polym. Sci., Polym. Chem. Ed. 2001, 39, 2520-2524; F. Tiarks, K. Landfester and M. Antonietti, “Silica nanoparticles as surfactants and fillers for latexes made by miniemulsion polymerization,” Langmuir 2001, 17, 5775-5780.

The encapsulated active agents may be produced in a size of about 1 nm to 500 nm, or in the form of microparticles having an average size ranging from about 5 nm to 5 μm. Active agents may be further encapsulated in mini- or micro-emulsions of suitable polymers. The term mini- or micro-emulsion may be understood as referring to dispersions comprising an aqueous phase, an oil or hydrophobic phase, and one or more surface active substances. Such emulsions may comprise suitable oils, water, one or several surfactants, optionally one or several co-surfactants and/or one or several hydrophobic substances. Mini-emulsions may comprise aqueous emulsions of monomers, oligomers or other pre-polymeric reactants stabilized by surfactants, which may be easily polymerized, and wherein the particle size of the emulgated droplets can be between about 10 nm and 500 nm or larger.

Mini-emulsions of encapsulated active agents can also be made from non-aqueous media, for example, formamide, glycol or non-polar solvents. Pre-polymeric reactants may comprise thermosets, thermoplastics, plastics, synthetic rubbers, extrudable polymers, injection molding polymers, moldable polymers, and the like, or mixtures thereof, including pre-polymeric reactants from which poly(meth)acrylics can be used.

Examples of suitable polymers for encapsulating the active agents can include, but are not limited to, homopolymers or copolymers of aliphatic or aromatic polyolefins such as polyethylene, polypropylene, polybutene, polyisobutene, polypentene; polybutadiene; polyvinyls such as polyvinyl chloride or polyvinyl alcohol, poly(meth)acrylic acid, polymethylmethacrylate (PMMA), polyacrylocyano acrylate; polyacrylonitril, polyamide, polyester, polyurethane, polystyrene, polytetrafluoroethylene; particularly preferred may be biopolymers such as collagen, albumin, gelatine, hyaluronic acid, starch, celluloses such as methylcellulose, hydroxypropyl cellulose, hydroxypropyl methylcellulose, carboxymethylcellulose phthalate; casein, dextranes, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate), shellac, and combinations of these homopolymers or copolymers; with the exception of cyclodextrine and derivatives thereof or similar carrier systems.

Other encapsulating materials that may be used include poly(meth)acrylate, unsaturated polyester, saturated polyester, polyolefines such as polyethylene, polypropylene, polybutylene, alkyd resins, epoxypolymers, epoxy resins, polyamide, polyimide, polyetherimide, polyamideimide, polyesterimide, polyesteramideimide, polyurethane, polycarbonate, polystyrene, polyphenole, polyvinylester, polysilicone, polyacetale, cellulosic acetate, polyvinylchloride, polyvinylacetate, polyvinylalcohol, polysulfone, polyphenylsulfone, polyethersulfone, polyketone, polyetherketone, polybenzimidazole, polybenzoxazole, polybenzthiazole, polyfluorocarbons, polyphenylenether, polyarylate, cyanatoester-polymere, or mixtures or copolymers of any of the foregoing.

In certain exemplary embodiments of the present invention, the polymers used to encapsulate the active agents may comprise mono(meth)acrylate-, di(meth)acrylate-, tri(meth)acrylate-, tetra-acrylate- and pentaacrylate-based poly(meth)acrylates. Examples for suitable mono(meth)acrylates are hydroxyethyl acrylate, hydroxyethyl methacrylate, hydroxypropyl methacrylate, hydroxypropyl acrylate, 3-chloro-2-hydroxypropyl acrylate, 3-chloro-2-hydroxypropyl methacrylate, 2,2-dimethylhydroxypropyl acrylate, 5-hydroxypentyl acrylate, diethylene glycol monoacrylate, trimethylolpropane monoacrylate, pentaerythritol monoacrylate, 2,2-dimethyl-3-hydroxypropyl acrylate, 5-hydroxypentyl methacrylate, diethylene glycol monomethacrylate, trimethylolpropane monomethacrylate, pentaerythritol monomethacrylate, hydroxy-methylated N-(1,1-dimethyl-3-oxobutyl)acrylamide, N-methylolacrylamide, N-methylolmethacrylamide, N-ethyl-N-methylolmethacrylamide, N-ethyl-N-methylolacrylamide, N,N-dimethylol-acrylamide, N-ethanolacrylamide, N-propanolacrylamide, N-methylolacrylamide, glycidyl acrylate, and glycidyl methacrylate, methyl acrylate, ethyl acrylate, propyl acrylate, butyl acrylate, amyl acrylate, ethylhexyl acrylate, octyl acrylate, t-octyl acrylate, 2-methoxyethyl acrylate, 2-butoxyethyl acrylate, 2-phenoxyethyl acrylate, chloroethyl acrylate, cyanoethyl acrylate, dimethylaminoethyl acrylate, benzyl acrylate, methoxybenzyl acrylate, furfuryl acrylate, tetrahydrofurfuryl acrylate and phenyl acrylate; di(meth)acrylates may be selected from 2,2-bis(4-methacryloxyphenyl)propane, 1,2-butanediol-diacrylate, 1,4-butanediol-diacrylate, 1,4-butanediol-dimethacrylate, 1,4-cyclohexanediol-dimethacrylate, 1,10-decanediol-dimethacrylate, diethylene-glycol-diacrylate, dipropyleneglycol-diacrylate, dimethylpropanediol-dimethacrylate, triethyleneglycol-dimethacrylate, tetraethyleneglycol-dimethacrylate, 1,6-hexanediol-diacrylate, Neopentylglycol-diacrylate, polyethyleneglycol-dimethacrylate, tripropyleneglycol-diacrylate, 2,2-bis[4-(2-acryloxyethoxy)phenyl]propane, 2,2-bis[4-(2-hydroxy-3methacryloxypropoxy)phenyl]propane, bis(2-methacryloxyethyl)N,N-1,9-nonylene-biscarbamate, 1,4-cycloheanedimethanol-dimethacrylate, and diacrylic urethane oligomers; tri(meth)acrylates may be selected from tris(2-hydroxyethyl)isocyanurate-trimethacrylate, tris(2-hydroxyethyl)isocyanurate-triacrylate, trimethylolpropane-trimethacrylate, trimethylolpropane-triacrylate or pentaerythritol-triacrylate; tetra(meth)acrylates may be selected from pentaerythritol-tetraacrylate, di-trimethylopropan-tetraacrylate, or ethoxylated pentaerythritol-tetraacrylate; suitable penta(meth)acrylates may be selected from dipentaerythritol-pentaacrylate or pentaacrylate-esters; as well as mixtures, copolymers or combinations of any of the foregoing.

For medical applications, biopolymers or acrylics may be used to encapsulate the active agents. In agricultural or other non-medical applications, acrylics, starch-based or cellulose-derived polymers may be selected as polymers for encapsulating the active agents.

Encapsulating polymer reactants may comprise polymerizable monomers, oligomers or elastomers such as polybutadiene, polyisobutylene, polyisoprene, poly(styrene-butadiene-styrene), polyurethanes, polychloroprene, natural rubber materials, gums such as gum arabica, locust bean gum, gum caraya, or silicone, and mixtures, copolymers or combinations of any of the foregoing. The active agents may be encapsulated in elastomeric polymers alone, or in mixtures of thermoplastic and elastomeric polymers, or in an alternating sequence of thermoplastic and elastomeric shells or layers.

The polymerization reaction for encapsulating the active agents can include any suitable conventional polymerization reaction, for example, a radical or non-radical polymerization, enzymatical or non-enzymatical polymerization, including poly-condensation reactions. The emulsions, dispersions or suspensions used may be in the form of aqueous, non-aqueous, polar or unpolar systems. By adding suitable surfactants, the amount and size of the emulgated or dispersed droplets can be adjusted as required.

The surfactants may be anionic, cationic, zwitter-ionic or non-ionic surfactants or any combinations thereof. Preferred anionic surfactants may include, but are not limited to, soaps, alkylbenzolsulphonates, alkansulphonates, olefinsulphonates, alkyethersulphonates, glycerinethersulphonates, α-methylestersulphonates, sulphonated fatty acids, alkylsulphates, fatty alcohol ether sulphates, glycerine ether sulphates, fatty acid ether sulphates, hydroxyl mixed ether sulphates, monoglyceride(ether)sulphates, fatty acid amide(ether)sulphates, mono- and di-alkylsulfosuccinates, mono- and dialkylsulfosuccinamates, sulfotriglycerides, amidsoaps, ethercarboxylicacid and their salts, fatty acid isothionates, fatty acid arcosinates, fatty acid taurides, N-acylaminoacid such as acyllactylates, acyltartrates, acylglutamates and acylaspartates, alkyloligoglucosidsulfates, protein fatty acid condensates, including plant derived products based on wheat; and alky(ether)phosphates.

Cationic surfactants suitable for encapsulation reactions in certain embodiments of the present invention may comprise quaternary ammonium compounds such as dimethyldistearylammoniumchloride, Stepantex® VL 90 (Stepan), esterquats, particularly quaternised fatty acid trialkanolaminester salts, salts of long-chain primary amines, quaternary ammonium compounds such as hexadecyltrimethyl-ammoniumchloride (CTMA-Cl), Dehyquart® A (cetrimoniumchloride, Cognis), or Dehyquart® LDB 50 (lauryldimethylbenzylammoniumchloride, Cognis).

Other preferred surfactants may include lecithin, poloxamers, i.e., block copolymers of ethylene oxide and propylene oxide, including those available from BASF Co. under the trade name pluronic®, including pluronic® F68NF, alcohol ethoxylate based surfactants from the TWEEN® series available from Sigma Aldrich or Krackeler Scientific Inc., and the like.

The active agent can be added before or during the start of the polymerization reaction and may be provided in the form of a dispersion, emulsion, suspension or solid solution, or as solution of the active agents in a suitable solvent or solvent mixture, or any mixtures thereof. The encapsulation process may comprise the polymerization reaction, optionally with the use of initiators, starters or catalysts, where an in-situ encapsulation of the active agents in polymer capsules, spheroids or droplets may occur. The solids content of the active agents in such encapsulation mixtures may be selected such that the solids content in the polymer capsules, spheroids or droplets is between about 10 weight % and about 80 weight % of active agent within the polymer particles.

Optionally, the active agents may also be added after completion of the polymerization reaction, either in solid form or in liquid form. The active agents can be selected from those compounds which are able to bind to the polymer spheroids or droplets, either covalently or non-covalently. The droplet size of the polymers and the solids content of active agents can be selected such that the solid content of the active agents is in the range of about 5 weight % to about 90 weight % with respect to the total weight of the encapsulated active agents.

In an exemplary embodiment of the present invention, the in-situ encapsulation of the active agents during the polymerization can be repeated at least once by addition of further monomers, oligomers or pre-polymeric agents after completion of a first polymerization/encapsulation step. By performing at least one repeated polymerization step in this manner, multilayer coated polymer capsules can be produced. Also, active agents bound to polymer spheroids or droplets may be encapsulated by subsequently adding monomers, oligomers or pre-polymeric reactants to overcoat the active agents with a polymer capsule. Repetition of such processes can produce multilayered polymer capsules comprising the active agent.

Any of the encapsulation steps described above may be combined with each other. In a preferred exemplary embodiment of the present invention, polymer-encapsulated active agents can be further coated with release-modifying agents.

In further exemplary embodiments of the present invention, the polymer-encapsulated active agents can be further encapsulated in vesicles, liposomes or micelles, or overcoatings. Surfactants that may be used for this purpose include, e.g., the surfactants described above, or compounds having hydrophobic groups which may include hydrocarbon residues or silicon residues, for example polysiloxane chains, hydrocarbon based monomers, oligomers and polymers or lipids or phosphorlipids or any combinations thereof, particularly glycerylester such as phosphatidyl-ethanolamine, phosphatidylcholine, polyglycolide, polylactide, polymethacrylate, polyvinylbuthylether, polystyrene, polycyclopentadienyl-methylnorbornene, polypropylene, polyethylene, polyisobutylene, polysiloxane, or any other type of surfactant.

Surfactants for encapsulating the polymer encapsulated active agents in vesicles, overcoats and the like may be selected from hydrophilic surfactants or surfactants having a hydrophilic residues or hydrophilic polymers such as polystyrensulfonicacid, poly-N-alkylvinylpyridinium-halogenide, poly(meth)acrylic acid, polyaminoacids, poly-N-vinylpyrrolidone, polyhydroxyethylmethacrylate, polyvinylether, polyethylenglycol, polypropylen-oxide, polysaccharides such as agarose, dextrane, starch, cellulose, amylase, amylo-pektin or polyethylenglycoles or polyethylenimines of a suitable molecular weight. Also mixtures from hydrophobic or hydrophilic polymer materials or lipid polymer compounds may be used to encapsulate the polymer-capsulated active agents in vesicles or for over-coating the polymer encapsulating active agents. The surfactant used may depend on the polymeric shell present.

The encapsulated active agents may also be chemically modified by functionalization with suitable linker groups or coatings which may be capable of reacting with the sol/gel forming components. For example, the encapsulated active agents may be functionalized with organosilane compounds or organo-functional silanes. Such compounds that may be used to modify the polymer-encapsulated active agents are described in more detail below.

The average particle size and particle size distribution of the encapsulated active agents in dispersed or suspended form may correspond to the average particle size and particle size distribution of the particles of finished encapsulated active agents, and thus they may have, e.g., a significant influence on the release properties of the drug delivery material produced. The average particle size and monodispersity of these encapsulated active agents can be characterized by, e.g., dynamic light scattering methods.

The polymer encapsulated active agents may be combined with a sol before subsequently being converted into a solid or semi-solid drug delivery material. In exemplary embodiments of the present invention, the sol can be prepared from any type of sol/gel forming components in a conventional manner. Suitable components and/or sols that are combined with the polymer encapsulated active agents may be selected based on the desired properties and requirements of the material to be produced.

The sol/gel forming components may comprise alkoxides, oxides, acetates, or nitrates of various metals, including but not limited to silicon, aluminum, boron, magnesium, zirconium, titanium, alkaline metals, alkaline earth metals, transition metals, platinum, molybdenum, iridium, tantalum, bismuth, tungsten, vanadium, cobalt, hafnium, niobium, chromium, manganese, rhenium, iron, gold, silver, copper, ruthenium, rhodium, palladium, osmium, lanthanum or lanthanides, as well as combinations thereof.

In certain exemplary embodiments of the present invention, the sol/gel forming components can comprise metal oxides, metal carbides, metal nitrides, metaloxynitrides, metalcarbonitrides, metaloxycarbides, metaloxynitrides, or metaloxycarbonitrides of the metals listed above, or any combinations thereof. These compounds, which may be in the form of colloidal particles, can be reacted with oxygen containing compounds such as, e.g., alkoxides to form a sol/gel, or they may be added as fillers if not provided in colloidal form.

In other exemplary embodiments of the present invention, the sols may be derived from at least one sol/gel forming component comprising alkoxides, metal alkoxides, colloidal particles, particularly metal oxides and the like. The metal alkoxides that may be used as sol/gel forming components can be conventional chemical compounds that may be used in a variety of applications. These compounds may have the general formula M(OR)ₓ where M is any metal from a metal alkoxide which may, e.g., hydrolyze and/or polymerize in the presence of water. R is an alkyl radical comprising between 1 and about 30 carbon atoms, which may be straight, chained or branched, and x can have a value equivalent to the metal ion valence. Metal alkoxides such as Si(OR)₄, Ti(OR)₄, Al(OR)₃, Zr(OR)₃ and Sn(OR)₄ may also be used. Specifically, R can be the methyl, ethyl, propyl or butyl radical. Further examples of suitable metal alkoxides can include Ti(isopropoxy)₄, Al(isopropoxy)₃, Al(sec-butoxy)₃, Zr(n-butoxy)₄ and Zr(n-propoxy)₄.

Sols can be made from silicon alkoxides such as tetraalkoxysilanes, wherein the alkoxy may be branched or straight chained and may contain 1 to 25 carbon atoms, e.g. tetramethoxysilane (TMOS), tetraethoxysilane (TEOS) or tetra-n-propoxysilane, as well as oligomeric forms thereof. Also suitable are alkylalkoxysilanes, wherein alkoxy is defined as above and alkyl may be a substituted or unsubstituted, branched or straight chain alkyl having about 1 to 25 carbon atoms, e.g., methyltrimethoxysilane (MTMOS), methyltriethoxysilane, ethyltriethoxysilane, ethyltrimethoxysilane, methyltripropoxysilane, methyltributoxysilane, propyltrimethoxysilane, propyltriethoxysilane, isobutyltriethoxysilane, isobutyltrimethoxy-silane, octyltriethoxysilane, octyltrimethoxysilane, which is commercially available from Degussa AG, Germany, methacryloxydecyltrimethoxysilane (MDTMS); aryltrialkoxysilanes such as phenyltrimethoxysilane (PTMOS), phenyltriethoxysilane, which is commercially available from Degussa AG, Germany; phenyltripropoxysilane, and phenyltributoxysilane, phenyl-tri-(3-glycidyloxy)-silane-oxide (TGPSO), 3-aminopropyltrimethoxysilane, 3-aminopropyl-triethoxysilane, 2-aminoethyl-3-aminopropyltrimethoxysilane, triaminofunctional propyltrimethoxysilane (Dynasylan® TRIAMO, available from Degussa AG, Germany), N-(n-butyl)-3-aminopropyltrimethoxysilane, 3-aminopropylmethyl-diethoxysilane, 3-glycidyl-oxypropyltrimethoxysilane, 3-glycidyloxypropyltriethoxy-silane, vinyltrimethoxysilane, vinyltriethoxysilane, 3-mercaptopropyltrimethoxy-silane, Bisphenol-A-glycidylsilanes; (meth)acrylsilanes, phenylsilanes, oligomeric or polymeric silanes, epoxysilanes; fluoroalkylsilanes such as fluoroalkyltrimethoxysilanes, fluoroalkyltriethoxysilanes with a partially or fully fluorinated, straight chain or branched fluoroalkyl residue of about 1 to 20 carbon atoms, e.g., tridecafluoro-1,1,2,2-tetrahydrooctyltriethoxysilane, or modified reactive flouroalkylsiloxanes which can be available from Degussa AG under the trademarks Dynasylan® F8800 and F8815; as well as any mixtures of the foregoing. Such sols may be easily converted into solid porous aerogels by drying.

In another exemplary embodiment of the present invention, the sol may be prepared from carbon-based nano-particles and organic alkaline or alkaline earth metal salts, e.g. formiates, acetates, propionates, malates, maleates, oxalates, tartrates, citrates, benzoates, salicylates, phtalates, stearates, phenolates, sulfonates, and amines, as well as acids, such as phosphorous acids, pentoxides, phosphates, or organo phosphorous compounds such as alkyl phosphonic acids. Other substances that may be used to form sols for bioerodible or dissolvable drug delivery materials may comprise sols made from magnesium acetate, calcium acetate, phosphorous acid, P₂O₅ as well as triethyl phosphite as a sol in ethanol or ethanediol, where biodegradable composites can be prepared from physiologically acceptable organic or inorganic components. For example, by varying the Ca/P-ratio, the degeneration rate of such composites can be adjusted. The molar ratio of Ca to P can be about 0.1 to 10, or preferably about 1 to 3.

In some exemplary embodiments of the present invention, the sols can be prepared from colloidal solutions which may comprise carbon-based nanoparticles in solutions, dispersions or suspensions in polar or nonpolar solvents, including aqueous solvents as well as cationically or anionically polymerizable polymers as precursors, such as alginate. By addition of suitable coagulators, e.g. inorganic or organic acids or bases, including acetates and diacetates, carbon-containing composite materials can be produced by precipitation or gel formation. Optionally, further additives can be added to adjust the properties of the resultant drug delivery material.

The sol/gel components used in the sols may also comprise colloidal metal oxides, including those colloidal metal oxides which are sufficiently stable to be combined with the other sol/gel components and the polymer-encapsulated active agents. Such colloidal metal oxides may include, but are not limited to, SiO₂, Al₂O₃, MgO, ZrO₂, TiO₂, SnO₂, ZrSiO₄, B₂O₃, La₂O₃, Sb₂O₅ and ZrO(NO₃)₂. SiO₂, Al₂O₃, ZrSiO₄ and ZrO₂ may be preferably selected. Further examples of the at least one sol/gel forming component include aluminum hydroxide sols or -gels, aluminum tri-sec-butylat, AlOOH-gels and the like.

Some of these colloidal sols may be acidic in the sol form and, therefore, when used during hydrolysis, it may not be necessary to add additional acid to the hydrolysis medium. These colloidal sols can also be prepared by a variety of methods. For example, titania sols having a particle size in the range of about 5 to 150 nm can be prepared by the acidic hydrolysis of titanium tetrachloride, by peptizing hydrous TiO₂ with tartaric acid, or by peptizing ammonia washed Ti(SO₄)₂ with hydrochloric acid. Such processes are described, for example, by Weiser in Inorganic Colloidal Chemistry, Vol. 2, p. 281 (1935). In order to preclude the incorporation of contaminants in the sols the alkyl orthoesters of the metals can be hydrolized in an acid pH range of about 1 to 3, in the presence of a water miscible solvent, wherein the colloid is present in the dispersion in an amount of about 0.1 to 10 weight percent.

In some exemplary embodiments of the present invention, the sols can comprise sol/gel forming components such as metal halides of the metals listed above, which may be reacted with oxygen-functionalized polymer-encapsulated active agents to form the desired sol. The sol/gel forming components may be oxygen-containing compounds, e.g., alkoxides, ethers, alcohols or acetates, which can be reacted with suitably functionalized polymer-encapsulated active agents. The encapsulated active agents can be dispersed into the sol by suitable blending methods such as stirring, shaking, extrusion, or the like.

Where a hydrolytic sol/gel-process is used to form the sol, the molar ratio of the added water to the sol/gel forming components, such as alkoxides, oxides, acetates, nitrides or combinations thereof, may be in the range of about 0.001 to 100, preferably from about 0.1 to 80, or more preferably from about 0.2 to 30.

In certain hydrolytric sol/gel processing procedures that may be used in accordance with exemplary embodiments of the present invention, the sol/gel components can be blended with the (optionally chemically modified) encapsulated active agents in the presence of water. Optionally, further solvents or mixtures thereof, and/or further additives may be added, such as surfactants, fillers and the like, as described in more detail below. The solvent may contain salts, buffers such as PBS buffer or the like, to adjust the pH value, the ionic strength, and similar properties. Further additives such as crosslinkers may be added, as well as catalysts for controlling the hydrolysis rate of the sol or for controlling the crosslinking rate. Such catalysts are also described in further detail below.

Non-hydrolytic sols may be made in a manner similar to that described above, but essentially in the absence of water.

When the sol is formed by a non-hydrolytic sol/gel-process or by chemically linking the components with a linker, the molar ratio of the halide to the oxygen-containing compound may be in the range of about 0.001 to 100, or preferably from about 0.1 to 140, even more preferably from about 0.1 to 100, or particularly preferably from about 0.2 to 80.

In nonhydrolytic sol/gel processes, metal alkoxides and carboxylic acids and their derivatives, or carboxylic acid functionalized polymer-encapsulated active agents may also be used. Suitable carboxylic acids can include acetic acid, acetoacetic acid, formic acid, maleic acid, crotonic acid, succinic acid, their anhydrids, esters and the like.

Non-hydrolytic sol/gel processing in the absence of water may be accomplished by reacting alkylsilanes or metal alkoxides with anhydrous organic acids, acid anhydrides or acid esters, or the like. Acids and their derivatives may be suitable as sol/gel components and/or to modify or functionalize the encapsulated active agents.

In certain exemplary embodiments of the present invention, the sol may be formed from at least one sol/gel forming component in an anhydrous sol/gel process, and the reactants can be selected from anhydrous organic acids, acid anhydrides or acid esters such as formic acid, acetic acid, acetoacetic acid, succinic acid, maleic acid, crotonic acid, acrylic acid, methacrylic acid, partially or fully fluorinated carboxylic acids, their anhydrides and esters, e.g. methyl- or ethylesters, or any mixtures of the foregoing. It may be preferable to use acid anhydrides in admixture with anhydrous alcohols, wherein the molar ratio of these components can determine the amount of residual acetoxy groups at the silicon atom of the alkylsilane used.

Acidic or basic catalysts may be applied, depending on the degree of crosslinking desired in the resulting sol or combination of sol and encapsulated active agents, particularly in hydrolytic sol/gel processes. Suitable inorganic acids include, for example, hydrochloric acid, sulfuric acid, phosphoric acid or nitric acid, as well as diluted hydrofluoric acid. Suitable bases include, for example, sodium hydroxide, ammonia and carbonate, as well as organic amines. Suitable catalysts in non-hydrolytic sol/gel processes may include anhydrous halide compounds, for example BCl₃, NH₃, AICl₃, TiCl₃ or mixtures thereof.

Solvents may be used to affect the hydrolysis in hydrolytic sol/gel processing steps in certain exemplary embodiments of the present invention, including water-miscible solvents such as water-miscible alcohols or mixtures thereof. Alcohols such as methanol, ethanol, n-propanol, isopropanol, n-butanol, isobutanol, t-butanol and lower molecular weight ether alcohols such as ethylene glycol monomethyl ether may be used. Small amounts of non-water-miscible solvents such as toluene may also be used. These solvents can also be used in polymer encapsulation reactions such as those described above.

In certain exemplary embodiments of the present invention, the sol or combination network may be further modified by the addition of at least one crosslinking agent to the sol, the encapsulated active agent, or the combination. The crosslinking agent may comprise, for example, isocyanates, silanes, diols, di-carboxylic acids, (meth)acrylates such as 2-hydroxyethyl methacrylate, propyltrimethoxysilane, 3-(trimethylsilyl)propyl methacrylate, isophorone diisocyanate, polyols, glycerine and the like. Biocompatible crosslinkers such as glycerine, diethylene triamino isocyanate and 1,6-diisocyanato hexane may also be used.

In further exemplary embodiments of the present invention, fillers can optionally be used to modify the pore sizes and the degree of porosity, if desired. Preferred fillers may include, but are not limited to, inorganic metal salts such as salts from alkaline and/or alkaline earth metals, preferably alkaline or alkaline earth metal carbonates, sulfates, sulfites, nitrates, nitrites, phosphates, phosphites, halides, sulfides, or oxides, as well as mixtures thereof. Other suitable fillers can include organic metal salts, e.g., alkaline or alkaline earth and/or transition metal salts, such as formiates, acetates, propionates, malates, maleates, oxalates, tartrates, citrates, benzoates, salicylates, phtalates, stearates, phenolates, sulfonates, and amines, as well as mixtures thereof.

Porosity in the resultant composite materials can be produced by treatment processes such as those described, e.g., in German Patent Publication No. DE 103 35 131 and in International Patent Application No. PCT/EP04/00077.

Other additives that can be used for controlling the conversion of the sols to gels and/or solid or semi-solid materials include, e.g., drying-control chemical additives such as glycerol, DMF, DMSO or any other suitable high boiling point or viscous liquids.

Solvents that can be used for the removal of fillers to affect porosity include, for example, (hot) water, diluted or concentrated inorganic or organic acids, bases and the like. Suitable inorganic acids include, for example, hydrochloric acid, sulfuric acid, phosphoric acid, nitric acid, or diluted hydrofluoric acid. Suitable bases include, for example, sodium hydroxide, ammonia, carbonate or organic amines. Suitable organic acids include, for example, formic acid, acetic acid, trichloromethane acid, trifluoromethane acid, citric acid, tartaric acid, oxalic acid, and mixtures thereof.

In exemplary embodiments of the present invention, coatings comprising the drug delivery materials produced in accordance with the processes described above may be applied as a liquid solution or dispersion or suspension of the combination in a suitable solvent or solvent mixture, with subsequent drying and/or evaporation of the solvent. Suitable solvents may comprise, for example, methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofurane, trimethyl hexanol, phenol, benzene, toluene, xylene; or water, any of which may be mixed with dispersants, surfactants or other additives and mixtures of the above-named substances.

The solvents listed herein can be used in the sol/gel process itself and/or in the encapsulation process, as described above. Solvents may comprise one or more of ethanol, isopropanol, n-propanol, dipropylene glycol methyl ether, butoxyisopropanol (1,2-propylene glycol-n-butyl ether), tetrahydrofurane, phenol, benzene, toluene, xylene, preferably ethanol, isopropanol, n-propanol and/or dipropylene glycol methyl ether.

Fillers, if used, can be partly or completely removed from the resultant drug delivery material depending on the nature and time of treatment with the solvent. A complete removal of the filler may be preferable for certain applications.

The combination of the sol and the encapsulated active agents formed as described above can be converted into a solid or semi-solid drug delivery material. Conversion of the combination into a gel, preferably an aerogel or xerogel, may be accomplished by, e.g., aging, curing, raising of pH, evaporation of solvent, or any other conventional method. The combination may be converted into the drug delivery material at room temperature, particularly where the components used may result in polymeric glassy composites, aerogels or xerogels.

The conversion step can be performed by drying the combination or the gel derived therefrom. In exemplary embodiments of the present invention, this drying step comprises a thermal treatment of the sol/combination or gel, in the range of about −200 C to +200 C, or preferably in the range of about −100° C. to 100° C., or more preferably in the range of about −50° C. to 100° C., or still more preferably about 0° C. to 90° C., or most preferably from about 10° C. to 80° C., or at about room temperature. Drying or aging may also be performed at any of the above temperatures under reduced pressure or in vacuo.

The conversion of the sol/combination into the solid or semi-solid material can be performed under various conditions. The conversion can be performed in different atmospheres, e.g., inert atmospheres such as nitrogen, SF₆, or noble gases such as argon, or any mixtures thereof, or it may be performed in an oxidizing atmosphere such as normal air, oxygen, carbon monoxide, carbon dioxide, or nitrogen oxide. Furthermore, an inert atmosphere may be blended with reactive gases, e.g. hydrogen, ammonia, C₁-C₆ saturated aliphatic hydrocarbons such as methane, ethane, propane and butene, mixtures thereof, or other oxidizing gases.

In exemplary embodiments of the present invention, the atmosphere used in any of the steps of the process may be substantially free of oxygen, particularly where oxygen sensitive components are used such as, e.g. organometallic compounds or certain alkoxides in non-hydrolytic sols. The oxygen content may preferably be below about 10 ppm, or more preferably below about 1 ppm.

In further exemplary embodiments of the present invention, high pressure may be applied to form the drug delivery material. The conversion step may be performed by drying under supercritical conditions, for example in supercritical carbon dioxide, which can lead to highly porous aerogel materials. Reduced pressure or a vacuum may also be employed to convert the sol/gel into the drug delivery material.

Suitable process conditions such as temperature, atmosphere and/or pressure may be selected based on the desired property of the final material and the components used to form the material.

By the incorporation of additives, fillers or functional materials, the properties of the materials produced can be influenced and/or modified in a controlled manner. For example, it may be possible to render the surface properties of the material hydrophilic or hydrophobic by incorporating inorganic nanoparticles or nanocomposites such as layer silicates.

Coatings or bulk materials comprising the encapsulated active agents may be processed or structured in a suitable way before or after conversion into the resultant drug delivery material by folding, embossing, punching, pressing, extruding, gathering, injection molding and the like, either before or after being applied to a substrate, molded or formed. In this way, certain structures of a regular or irregular type can be incorporated into the active agent-containing coating comprising the drug delivery material.

The combination materials can be further processed by conventional techniques, e.g., they can be used to build molded paddings and the like, or to form coatings on substrates. Molded paddings can be produced in almost any desired form. The molded paddings may be in the form of pipes, bead-mouldings, plates, blocks, cuboids, cubes, spheres or hollow spheres or any other three-dimensional structure, which may be, for example longish, circle-shaped, polyhedral, e.g. triangular, bar-shaped, plate-shaped, tetrahedral, pyramidal, octahedral, dodecahedral, icosahedral, rhomboidal, prismatic, or in round shapes such as ball-shaped, spheroidal or cylindrical, lens-shaped, ring-shaped, honeycomb-shaped, and the like.

The material can be brought into the desired form by applying any appropriate conventional shaping technique including, but not limited to, casting processes such as sand casting, shell molding, full mold processes, die casting, centrifugal casting, or by pressing, sintering, injection molding, compression molding, blow molding, extrusion, calendaring, fusion welding, pressure welding, jiggering, slip casting, dry pressing, drying, firing, filament winding, pultrusion, lamination, autoclave, curing or braiding.

Coatings formed from sols/combinations may be applied in liquid, pulpy or pasty form, for example, by painting, furnishing, phase-inversion, dispersing, atomizing or melt coating, extruding, slip casting, dipping, or as a hot melt. If the combination is in a solid or semi-solid state, it may be applied as a coating onto a suitable substrate by, e.g., powder coating, flame spraying, sintering or the like. Dipping, spraying, spin coating, ink-jet-printing, tampon and microdrop coating or 3D printing may also be used.

Combination sols or gels can be processed by any appropriate conventional technique. Preferred techniques may include folding, stamping, punching, printing, extruding, die casting, injection molding, reaping, and the like. Coatings may also be obtained by a transfer process, in which the combination gels are applied to a substrate as a lamination. The coated substrate can be cured, and subsequently the coating can be released from the substrate to be thermally treated. The coating of the substrate can be formed by using suitable printing procedures, e.g. gravure printing, scraping or blade printing, spraying techniques, thermal laminations or wet-in-wet laminations. A plurality of thin layers may be successively applied to provide a more uniform and thicker coating, and/or to more precisely control a correct dosing of the active agent.

By applying the transfer procedure described above, it is possible to form multi-layer gradient films by using different material layers and/or different sequences of layers. Conversion of these multilayer coatings into a composite material can result in gradient materials, wherein the density, the release properties and/or the active agent concentration in the material may vary from place to place. Non-linear release profiles of the active agents may be achieved in this manner, which may be desirable for specific drugs and/or applications.

In another exemplary embodiment of the present invention, the combination may be dried or thermally treated and commuted by conventional techniques, for example, by grinding in a ball mill or roller mill and the like. The commuted material can be used as a powder, a flat blank, a rod, a sphere, a hollow sphere in different grainings, and the like, and can be further processed by conventional techniques to form granulates or extrudates in various forms.

Additional processing options can include, but are not limited to, the formation of powders by other conventional techniques such as spray-pyrolysis or precipitation, and the formation of fibers by spinning-techniques, such as gel-spinning.

The porosity and the pore sizes may also be varied over a wide range, simply by varying the components in the sol and/or by varying the particle size of the encapsulated active agents, which may be used to control the release properties. Depending on the active agents used, their in vivo and/or in vitro release can be controlled by adjusting suitable pore sizes in the sol/gel matrix.

Furthermore, by suitable selection of components and processing conditions, bioerodible coatings, or coatings and materials which are dissolvable or may be peeled off from substrates in the presence of physiologic fluids can be produced. For example, coatings comprising the drug delivery material may be used in coronary implants such as stents, wherein the coating may further comprise, in addition to the active agent, an encapsulated or unencapsulated marker such as a metal compound having signaling properties, and which thus may produce signals detectable by physical, chemical or biological detection methods such as x-ray, nuclear magnetic resonance (NMR), computer tomography methods, scintigraphy, single-photon-emission computed tomography (SPECT), ultrasonic methods, radiofrequency (RF) methods, and the like. Metal compounds that can be used as markers may also be encapsulated in a polymer shell together with or independent of the active agents, and thus they can be prevented from interfering with the implant material, which may also be a metal, where such interference can often lead to electrocorrosion or related problems.

Coated implants may be produced with drug delivery coatings, wherein the coating remains permanently on the implant. In one exemplary embodiment of the present invention, the coating may be slowly or rapidly dissolved or peeled off from the stent after implantation under physiological conditions, thus allowing for a controlled release of the active agent. Additionally, with a suitable selection of the encapsulating material, the release of the active agents can be further modified, e.g. by using dissolvable or swellable encapsulating materials which can controllably release the active agent in the presence of water, solvents or physiologic fluids.

Further options for modification of the release rate of the encapsulated active agents from the drug delivery materials may include, for example, the incorporation of fillers such as porogenous fillers, hydrophilic or hydrophobic fillers, which, in the presence of solvents such as water or physiological fluids, can influence the elution rate of the encapsulated active agents. Also, with the incorporation of such fillers or surface active substances, the surface tension at the interfaces between encapsulated active agents and the sol/gel matrix can be modified, which may also directly affect the release rate of the active agents.

The active agents may be eluted from the drug delivery materials by eluting or releasing the complete capsules or polymeric shells, which may subsequently be dissolved or degraded. Alternatively, the shell of an encapsulated active agent may be degraded under the influence of physiological fluids or solvents within the sol/gel matrix, and the active agents may then be directly released from the drug delivery materials.

The drug delivery materials formed by the exemplary processes described herein may have specific advantages when compared to conventional drug delivery systems where the active agent is simply dispersed in the sol/gel matrix without encapsulation. The encapsulation of the active agents can form a separation of the active agents in a substantially inert surrounding, so that interactions with the sol/gel materials or with substances used during the sol/gel process, such as solvents, salts and the like are avoided. Such interactions may, in case of sensitive active agents, lead to degradation reactions or even inactivation of the active agents. For example, proteins may be denaturated when contacted by sol/gel components. This can be avoided by encapsulating the proteins in polymeric or surfactant shells as described herein. Also, the formation of intermediates of polycyclic active agents with sol/gel components can be avoided by the encapsulation process.

Furthermore, it can be possible to adjust the release kinetics of the active agent from the inventive material independent of the sol/gel material used by suitable selection of the encapsulation material, the thickness of the encapsulation shell, and the like. By using hydrophilic or hydrophobic encapsulation polymers, the release characteristics may be suitably influenced and adapted to the media where the release occurs. The number of side chains of cross-linked or branched polymers used to form the encapsulation materials may also have a direct influence on the release kinetics.

The combination of sol/gel materials that can be used, particularly those which are bioresorbable or biodegradable, my advantageously allow for the incorporation of fillers and the simultaneous incorporation of the encapsulated active agents, which provides new possibilities for individually adjusting the release rate and the release kinetics of agents from the drug delivery materials. These advantages may be particularly beneficial in coatings.

The method of producing drug delivery materials described herein may be simpler and more reproducible or consistent compared to conventional methods, since the formation of active agents in polymer capsules can be done independently of the formation of the sol/gel matrix. The release kinetics of the active agent can also be decoupled from the degradation kinetics of a resorbable implant or coating of the implant itself. This advantage may be particularly relevant if the substrate or carrier of the drug delivery material is resorbed faster in vivo (as is the case with some magnesium or zinc alloys, for example), and a different release kinetic or release profile of the active agent is desired. In a further exemplary embodiment of the present invention, a combined first carrier/second carrier mechanism may be created, i.e., the sol/gel matrix used in the drug delivery materials can be the first carrier that transports the encapsulated active agents, and the shells/capsules carrying the encapsulated active agents may act as the second carrier, controlling the release of the active agent itself.

This first carrier/second carrier mechanism may be particularly advantageous if the implant comprising the drug delivery material can only reach a specific compartment of an organ or organism (e.g., an endoluminal coronary stent may only be able to reach the intra-vascular space). The second carrier in the drug delivery material, i.e., the polymer encapsulated active agent, may then have access via physiological pathways to another compartment (for example, the extra vascular space). This mechanism may be particularly desirable in certain local drug delivery applications, if the drug itself is not enriched primarily in a compartment where the implant is placed. An example of this can be the use of hydrophilic proteins as the active agents, where these proteins can be transported from the intravascular space to the local surrounding extravascular space.

The drug delivery materials described herein can be used for the production or coating of medical implants such as coronary stents comprising corrosive materials including, for example, implants comprising magnesium or zinc alloys, bone grafts made of biocorrosive material or degradable material and the like. It may be preferable to use the drug delivery material for the manufacture of medical implants for replacement of organs or tissue, e.g. bone grafts, prostheses and the like, where the implants may be manufactured totally or partially from the drug delivery material.

### EXAMPLES

The present invention will now be further described by way of the following non-limiting examples. Analyses and parameter determination in these examples were performed by the following methods:

Particle sizes are provided as mean particle sizes, as determined on a CIS Particle Analyzer (Ankersmid) by the TOT-method (Time-Of-Transition), X-ray powder diffraction, or TEM (Transmission-Electron-Microscopy). Average particle sizes in suspensions, emulsions or dispersions were determined by dynamic light scattering methods. Average pore sizes of the materials were determined by SEM (Scanning Electron Microscopy). Porosity and specific surface areas were determined by N₂ or He absorption techniques, according to the BET method.

### Example 1

20 mg of poly(DL-lactide-co-glycolide) and 2 mg of paclitaxel were added to 3 ml of acetone. The resulting solution was added at a constant flow rate of 10 ml per minute to a stirred (400 rpm) solution of 0.1% poloxamer 188 surfactant (pluronic® F68, available from BASF Co., N.J., US) in 0.05 M PBS buffer (phosphate-buffered saline), and the resulting colloidal suspension was stirred for an additional 3 hours under a slight vacuum to partially evaporate the solvent. The mixture was then dried for 14 hours in vacuo. The resulting nano-particles comprising encapsulated paclitaxel had a mean particle size of about 140 to 170 nm.

300 gm of tetraethylorthosilane TEOS (obtained from Degussa AG, Germany) in 300 g of deionized water with 1 g of 1N HCl as a catalyst were stirred for 30 minutes at room temperature in a glass vessel to produce a homogeneous sol. 5 ml of this sol were combined with 2 ml of a 5 mg per ml suspension of the above-produced capsules in ethanol, and 0.1 wt. % of lecithin was added as a surfactant. The suspension was stirred for 6 hours at room temperature and subsequently sprayed onto a commercially available coronary stent obtained from Fortimedix Co. (KAON 18.5 mm). The sprayed layer was dried for two hours at room temperature and had a gel-like, semi-solid consistency. The resulting layer had a thickness of about 3 μm.

3 coronary stents coated as described above were incubated in an Eppendorf-cup while shaking (75 rpm) at 37.5° C. for 30 days in 4 ml of PBS buffer, and the supernatant buffer solution was removed once per day and replaced by fresh buffer. In the removed supernatant solution, the amount of released paclitaxel was determined via HPLC. After 1 day about 30% of the total amount of the paclitaxel present in the coating was released, increasing to about 50% after 5 days, and about 70% after 30 days.

### Example 2

In this example, encapsulated paclitaxel was prepared in accordance with the procedure as outlined above in Example 1.

300 g tetramethylorthosilane (TMOS) (Degussa AG) were combined with 300 g of deionized water, 3 g of TWEEN®20 (polyoxyethylene sorbitan monolaurate, obtained from Sigma Aldrich) as the surfactant, and 1 ml of 1N HCl as a catalyst were added, and the mixture was stirred for 30 minutes at room temperature in a glass vessel in order to produce a homogeneous sol. 5 ml of this sol and 2 ml of a 5 mg per ml suspension of the encapsulated paclitaxel in ethanol were combined, stirred for 6 hours at room temperature and subsequently aged for five days at room temperature in 2 ml Eppendorf-cups. The material was then dried in vacuo. The aerogels so obtained had the form of a spheroidal powder of milky appearance.

The aerogels had biodegradable properties and released the paclitaxel in a controlled manner. The release of the paclitaxel was determined as follows: The aerogel particles were incubated in 4 ml of PBS buffer while shaking at 75 rpm for thirty days at 37.5° C. A 1.2 ml volume of the aerogel particles was used. The buffer supernatant was removed daily and replaced by fresh buffer. The amount of paclitaxel released in the supernatant was determined via HPLC. The average release rate of paclitaxel was relatively constant at about 6 to 8 weight % of the total amount present per day.

### Example 3

Encapsulated paclitaxel was prepared in accordance with Example 1. A homogenous sol was prepared from 100 ml of a 20 weight % solution of magnesium acetate tetrahydrate (Mg(CH₃COO)₂*4H₂O) in ethanol and 10 ml of a 10% nitric acid, which was then stirred for three hours at room temperature. 4 ml of tetraethylorthosilane TEOS (obtained from Degussa AG) were added to the sol and the mixture was stirred for another two hours at room temperature (20 rpm). 5 ml of the sol was combined with 2 ml of a 5 mg per ml suspension of the encapsulated paclitaxel in ethanol, 0.1 weight % lecithin was added as a surfactant, and the combination was stirred for 6 hours at room temperature and subsequently sprayed onto a commercially available coronary stent of Fortimedix Co. (KAON 18.5 mm). The homogeneous layer was dried for 10 minutes at about 40° C. in a hot air stream.

The coated coronary stents were incubated in an Eppendorf-cup in 4 ml of PBS buffer while shaking at 75 rpm for 30 days at 37.5° C. The buffer supernatant was removed daily and was replaced by fresh buffer. The amount of the released paclitaxel in the supernatant was determined by HPLC. 10 weight % of the paclitaxel was released after the first day, 15% was released after 5 days, and 40% of the total amount of the paclitaxel was released after 30 days.

### Example 4

Encapsulated paclitaxel was prepared as described in Example 1 above. A homogeneous sol was prepared from 100 ml of a 20 weight % solution of magnesium acetate tetrahydrate in ethanol and 10 ml of a 10% nitric acid at room temperature and stirring for 3 hours. 4 ml of TEOS (obtained from Degussa AG) were added and the mixture was stirred for further 2 hours at room temperature (20 rpm). 5 ml of the resulting gel was combined with 2 ml of a 5 mg per ml suspension of paclitaxel capsules in ethanol, 2 weight % of lecithin as a surfactant, and 5 weight % of polyethylene glycol PEG 400 as a filler. The combination was stirred for 6 hours at room temperature and aged for 5 days in 2 ml Eppendorf-cups. The material was then dried in vacuo. The resulting gel comprised spheroidal particles having a milky appearance.

The aerogels had biodegradable and controlled release properties. The release rate was determined by incubating the aerogels in 4 ml of PBS buffer, while shaking at 75 rpm for thirty days at 37.5° C. The buffer supernatant was removed daily and replaced by fresh buffer. The amount of paclitaxel released into the supernatant was determined via HPLC. The average release rate of paclitexal in this example was constant at about 2% of the total amount per day.

Having thus described in detail several exemplary embodiments of the present invention, it is to be understood that the invention described above is not to be limited to particular details set forth in the above description, as many apparent variations thereof are possible without departing from the spirit or scope of the present invention. The embodiments of the present invention are disclosed herein or are obvious from and encompassed by the detailed description. The detailed description, given by way of example, is not intended to limit the invention solely to the specific embodiments described.

The foregoing applications and all documents or publications cited therein or during their prosecution (“appln. cited documents”) and all documents cited or referenced in the appln. cited documents, and all documents, references and publications cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference in their entirety, and may be employed in the practice of the invention. Citation or identification of any document in this application is not an admission that such document is available as prior art to the present invention.

It is noted that in this disclosure, and particularly in the claims, terms such as “comprises,” “comprised,” “comprising” and the like can have the meaning attributed to them in U.S. Patent law; e.g., they can mean “includes,” “included,” “including” and the like; and that terms such as “consisting essentially of” and “consists essentially of” can have the meaning ascribed to them in U.S. Patent law, e.g., they allow for elements not explicitly recited, but exclude elements that are found in the prior art or that affect a basic or novel characteristic of the invention.

What is claimed is:

1. A method for manufacturing a drug delivery material comprising:
(a) encapsulating at least one of a biologically active agent or a therapeutically active agent in a shell to form a first composition;
(b) combining the first composition with a sol to form a second composition; and
(c) converting the second composition into at least one of a solid drug delivery material or a semi-solid drug delivery material.

2. The method of claim 1, wherein the shell comprises a polymer.

3. The method of claim 1, further comprising forming the sol using a hydrolytic sol/gel-process in the presence of water.

4. The method of claim 1, further comprising forming the sol using a non-hydrolytic sol/gel-process in the absence of water.

5. The method of claim 1, wherein the at least one of a biologically active agent or a therapeutically active agent is a therapeutically active agent that is capable of providing at least one of a direct therapeutic effect, a direct physiological effect, a direct pharmacological effect, an indirect therapeutic effect, an indirect physiological effect, or an indirect pharmacological effect in at least one of a human organism or an animal organism.

6. The method of claim 5, wherein the at least one of the biologically active agent or the therapeutically active agent is at least one of a medicament, a drug, a pro-drug, a drug, or a pro-drug comprising at least one targeting group.

7. The method of claim 1, wherein the shell comprises at least one of poly(meth)acrylate, poly(DL-lactide-co-glycolide), poly(D,L-lactide), polyglycolide, an unsaturated polyester, a saturated polyester, a polyolefine, polyethylene, polypropylene, polybutylene, an alkyd resin, an epoxy-polymer, an epoxy resin, a polyamide, a polyimide, a polyetherimide, a polyamideimide, a polyesterimide, a polyesteramideimide, polyurethane, a polycarbonate, a polystyrene, a polyphenole, a polyvinylester, a polysilicone, a polyacetale, a cellulosic acetate, a polyvinylchloride, a polyvinylacetate, a polyvinylalcohol, a polysulfone, a polyphenylsulfone, a polyethersulfone, a polyketone, a polyetherketone, a polybenzimidazole, a polybenzoxazole, a polybenzthiazole, a polyfluorocarbon, a polyphenylenether, a polyarylate, a cyanatoester-polymer, or a copolymer of any of the foregoing.

8. The method of claim 1, wherein the shell comprises at least one of poly(D,L-lactide), polyglycolide, poly(DL-lactide-co-glycolide), or polymethylmethacrylate (PMMA).

9. The method of claim 7, wherein step (a) comprises using at least one of a dispersion process, a suspension process, an emulsion-polymerization process, an enzymatic process, or a radical polymerization process.

10. The method of claim 9, further comprising providing the at least one of the biologically active agent or the therapeutically active agent at least one of before or during a start of the at least one of the dispersion process, the suspension process, the emulsion-polymerization process, the enzymatic process, or the radical polymerization process.

11. The method of claim 1, wherein the shell comprises a plurality of layers of organic material.

12. The method of claim 1, further comprising functionalizing the at least one of a biologically active agent or a therapeutically active agent by a chemical modification with at least one of a linker group or a coating that is capable of reacting with sol/gel forming components.

13. The method of claim 1, further comprising preparing the sol from at least one sol/gel forming component comprising at least one of an alkoxides, a metal alkoxide, a metal oxide, a metal acetate, a metal nitrate, or a metal halide, wherein the at least one of the metal alkoxide, the metal oxide, the metal acetate, the metal nitrate, or the metal halide metal further comprises at least one of silicon, aluminum, boron, magnesium, zirconium, titanium, alkaline metals, alkaline earth metals, or transition metals, platinum, molybdenum, iridium, tantalum, bismuth, tungsten, vanadium, cobalt, hafnium, niobium, chromium, manganese, rhenium, iron, gold, silver, copper, ruthenium, rhodium, palladium, osmium, lanthanum or a lanthanide.

14. The method of claim 13, wherein the at least one sol/gel forming component comprises at least one of a silicon alkoxide, a tetraalkoxysilane, an oligomeric form of a silicon alkoxide, an alkylalkoxysilanes, an aryltrialkoxysilanes, an aminoalkylalkoxysilane, an alkenylalkoxysilane, a bisphenol-A-glycidylsilane, a (meth)acrylsilane, an epoxysilanes, or a fluoroalkylalkoxysilane.

15. The method of claim 1, further comprising forming the sol in the presence of an organic solvent, wherein the organic solvent content of the sol is between about 0.1% and 90%.

16. The method of claim 1, further comprising forming the sol in the presence of an organic solvent, wherein an organic solvent content of the sol is between about 1% and 90%.

17. The method of claim 1, further comprising forming the sol in the presence of an organic solvent, wherein an organic solvent content of the sol is between about 5% and 90%.

18. The method of claim 1, further comprising forming the sol in the presence of an organic solvent, wherein an organic solvent content of the sol is between about 20% and 70%.

19. The method of claim 1, further comprising adding at least one additive to at least one of the first composition, the sol, or the second composition, wherein the at least one additive comprises at least one of a biologically active compound, a therapeutically active compound, a filler, a surfactant, an acid, a base, a crosslinker, a pore-forming agent, a plasticizer, a lubricant, a flame resistant composition, a glass, a glass fiber, a carbon fiber, cotton, a fabric, a metal powder, a metal compound, silicon, a silicon oxide, azeolite, a titanium oxide, a zirconium oxide, an aluminum oxide, an aluminum silicate, talcum, graphite, soot, a phyllosilicate, a drying-control chemical additive, glycerol, DMF, or DMSO.

20. The method of claim 1, wherein step (c) comprises at least one of hydrolyzing the second composition, aging the second composition, crosslinking the second composition, or drying the second composition.

21. The method of claim 1, wherein step (c) comprises drying the second composition by a thermal treatment in the range of about −200° C. to 100° C.

22. The method of claim 21, wherein step (c) is performed under at least one of a reduced pressure or a vacuum.

23. The method of claim 1, further comprising adding at least one crosslinking agent to at least one of the first composition, the sol or the second composition, wherein the crosslinking agent comprises at least one of an isocyanate, a silane, a (meth)acrylate, 2-hydroxyethyl methacrylate, propyltrimethoxysilane, 3-(trimethylsilyl)propyl methacrylate, isophoron diisocyanate, HMDI, diethylenetriaminoisocyanate, or 1,6-diisocyanatohexane.

24. The method of claim 1, further comprising adding at least one filler to at least one of the first composition, the sol or the second composition, wherein the at least one filler is incapable of reacting with the other components of the sol.

25. The method of claim 24, wherein the at least one filler is a polymer encapsulated fullerene.

26. The method of claim 24, further comprising at least partially removing the filler from the solid drug delivery material.

27. A drug delivery material comprising at least one portion formed by:
(a) encapsulating at least one of a biologically active agent or a therapeutically active agent in a shell to form a first composition;
(b) combining the first composition with a sol to form a second composition; and
(c) converting the second composition into at least one of a solid drug delivery material or a semi-solid drug delivery material,
wherein the drug delivery material is in the form of at least one of a coating or a bulk material.

28. The drug delivery material of claim 27, wherein the material is at least one of dissolvable in physiological fluids or includes bioerodible properties in a presence of physiological fluids.

29. The drug delivery material of claim 27, wherein the material has a form of an implant.

30. The drug delivery material of claim 29, wherein the implant is capable of providing a sustained release of the at least one of the biologically active agent or the therapeutically active agent when inserted into at least one of a human body or an animal body.
